(19) United States
(12) Patent Application Publication
(10) Pub. No.: US 2004/0197908 A1
(43) Pub. Date: Oct. 7, 2004

(54) Artificial cartilage

(76) Inventors: Minoru Ueda, Nagoya-shi (JP); Katsumi Ebisawa, Nagoya-shi (JP); Shuhei Torii, Nagoya-shi (JP); Kenichiro Hata, Nagoya-shi (JP); Kunihiko Okada, Nagoya-shi (JP); Hideaki Kagami, Nagoya-shi (JP); Hideto Watanabe, Aichi-gun (JP); Koji Kimata, Aichi-gun (JP)

Correspondence Address: RADER FISHMAN & GRAUER PLLC, LION BUILDING, 1233 20TH STREET N.W., SUITE 501, WASHINGTON, DC, 20036, US

(21) Appl. No.: 10/674,414

(22) Filed: Oct. 1, 2003

(30) Foreign Application Priority Data
Apr. 2, 2003 (JP) 2003-098989

(51) Int. Cl. C12N 5/00; C12N 5/02

(52) U.S. Cl. 435/395; 435/375

(57) ABSTRACT

The following are provided: a method for manufacturing artificial cartilages characterized in that undifferentiated mesenchymal cells are cultured in a cartilage differentiation inducing medium, and the cells are irradiated with ultrasound, as a method in which chondrocytes can be obtained in a short time in an amount sufficient for performing the treatment of cartilage disorder with surgical operation when a conservative therapy such as limitation of movement exhibits no effect, among various treatments of cartilage disorder; an artificial cartilage that has been produced by the method; and an apparatus for manufacturing artificial cartilages comprising a culture vessel containing cultured undifferentiated mesenchymal cells, an ultrasound transducer for applying ultrasound to the vessel, a control means for controlling the ultrasound, and a holding water-tank for installing the ultrasound transducer and the culture vessel in the holding water-tank in such a state that they are in contact with each other.

### TECHNICAL FIELD

The present invention relates to a differentiation-inducing effect enhanced by physical-mechanical stimulation applied on undifferentiated cells. More particularly, it relates to an artificial cartilage which is produced through the acceleration of differentiation of the undifferentiated cells to chondrocytes by irradiating the undifferentiated cells with ultrasound, in order to use it for the therapy such as chondrocyte transplantation, cartilaginous tissue transplantation, osseocartilaginous cell transplantation or osseocartilaginous tissue transplantation, and a method and an apparatus for manufacturing the artificial cartilage.

### BACKGROUND ART

For the treatment of cartilage disorder, a conservative treatment such as limitation of movement is mainly adopted. But, the conservative treatment has often no effect on the cartilage disorder, and a therapy with a surgical operation is used in such cases.

For the surgical operation of a cartilage disorder site, a method wherein cartilaginous tissue or osseocartilaginous tissue is collected from a non-damaged site, and the tissue is transplanted (Hangody et al.: Foot Ankle Int Oct: 18 (10), 628-34, 1997), a method wherein chondrocytes collected from a non-damaged site are subjected to an in vitro culture to proliferate them, and the proliferated chondrocytes are transplanted to a damaged site (Brittberg et al.: New Eng. J. Med. 331, 889-895, 1994) or the like is adopted. However, in the method wherein cartilaginous tissue is collected from a non-damaged site, the amount of the collection is limited, and also in the method wherein the collected chondrocytes are proliferated by the in vitro culture, it requires a long time to obtain a sufficient amount of chondrocytes.

On the other hand, it has been reported that the irradiation of chondrocytes with ultrasound during culturing enhances the biosynthesis of cartilage substrate (Parvizi et al.: J. Orthop. Res., 17(4):488-94, 1999; Nishikori et al.: J. Biomed. Master Res., 59(2) 201-206, 2002; and Zi-Jun Zhang et al.: 4^{th} Symposium International Cartilage Repair Society, Tront, 15-18/June, 2002).

Regarding ultrasound which brings about such effect, Duarte et al. performed studies for applying it to bone fracture therapy (U.S. Pat. No. 4,530,360), and subsequently an ultrasound-accelerated fracture healing device, SAFHS™, was developed by Exogen Inc. in USA. It was confirmed that the ultrasound-accelerated fracture healing device, SAFHS™, had healing-acceleration effects on fractures of the tibial diaphysis (Heckman et al., J. Bone and Joint Surg., 76A: 25-34, 1994), on fractures of the distal radius (Kristiansen et al., J. Bone and Joint Surg., 79A:961-973, 1997) and so on in clinical trials.

Further, Pittenger et al. reported that undifferentiated mesenchymal cells can be differentiated into fat-cells, chondrocytes and osteocytes in vitro, and a medium composition which can induce the differentiation into said three kinds of cells were reported (SCIENCE 284(2): 143-147, 1999, WO 98/32333). Further, it was reported that bone differentiation is accelerated by the application of ultrasound by using an ultrasound-accelerated fracture healing device (SAFHS™) during the culture of undifferentiated mesenchymal cells in a bone differentiation inducing medium (Okada et al.: The 4^{th} Japanese. Society for Tissue Engineering, at Kawasaki, 6-7 July, 2001).

### DISCLOSURE OF THE INVENTION

The present invention provides a method in which chondrocytes can be obtained in a short time in an amount sufficient for performing the treatment of cartilage disorder with surgical operation when a conservative therapy such as limitation of movement exhibits no effect, among various treatments of cartilage disorder, and an apparatus therefor.

The inventors of the present invention pursued zealous studies on such problems, and they found that the application of mechanical and dynamic stimulation, that is, ultrasound irradiation to undifferentiated mesenchymal cells during culture accelerates cartilage differentiation. Thus, they reached the present invention.

That is, the present invention provides a method for manufacturing artificial cartilage, based on a cell differentiation inducing method which is characterized in that undifferentiated cells are irradiated with ultrasound, and specifically a manufacturing method which is characterized in that undifferentiated mesenchymal cells are cultured in a cartilage differentiation inducing medium, and ultrasound is applied on the undifferentiated cells. Especially, the present invention provides a method for manufacturing artificial cartilage, wherein the manufacturing method is characterized in that the ultrasound to be applied is pulsed ultrasound; the ultrasound has a frequency of 20 kHz to 10 MHz, a burst width of 10 μsec to 1 msec, a repetition rate of 5 Hz to 10 kHz, and an ultrasound intensity of 5-120 mW/cm²; and preferably, the ultrasound has a frequency of 1.5 MHz, a burst width of 200 μsec, a repetition rate of 1.0 kHz and an ultrasound intensity of 5-120 mW/cm².

Further, the present invention provides an apparatus for manufacturing artificial cartilage, wherein the apparatus comprises a culture vessel containing cultured undifferentiated mesenchymal cells, an ultrasound transducer for applying ultrasound to the vessel, a control means for controlling the ultrasound, and a holding water-tank for installing the ultrasound transducer and the culture vessel in the holding water-tank in such a state that they are in contact with each other.

Furthermore, the present invention provide an apparatus for manufacturing artificial cartilage, wherein the apparatus is characterized in that the control means is a means for controlling the ultrasound transducer so that ultrasound pulse is output from it, and the pulsed ultrasound has a frequency of 20 kHz to 10 MHz, a burst width of 10 μsec to 1 msec, a repetition rate of 5 Hz to 10 kHz, and an ultrasound intensity of 5-120 mW/cm².

### BRIEF DESCRIPTION OF THE DRAWINGS

FIG. 1 is a block diagram of an apparatus for manufacturing artificial cartilage of the present invention.

FIG. 2 shows external appearances of an ultrasound-generating unit of the apparatus for manufacturing artificial cartilage of the present invention.

FIG. 3 shows the results of alcian blue staining of pellets.

FIG. 4 shows the results of immunostaining of aggrecans.

FIG. 5 shows the results of pellet sizes in each culture group.

FIG. 6 shows the results of amounts of DNA in each culture group.

FIG. 7 shows the results of total amounts of protein in each culture group.

FIG. 8 shows the results of amounts of aggrecan in each culture group.

FIG. 9 shows the correlation of ultrasound intensities and an amount of aggrecan.

### BEST MODE FOR CARRYING OUT THE INVENTION

The present invention relates to a method for manufacturing artificial cartilage by the differentiation induction of undifferentiated cells, especially by the differentiation induction of undifferentiated mesenchymal cells derived from bone marrow to chondrocytes, and an ultrasound irradiation apparatus to be used for the method, that is, an apparatus for manufacturing artificial cartilage.

The ultrasound transducer for radiating ultrasound is made of a piezoelectric ceramic. Electric signals produced by an ultrasonic wave synthesizing apparatus are given to the piezoelectric ceramic of the transducer, and the transducer generates vibration (ultrasound) in the longitudinal direction on its surface by using the characteristics that microstrains are generated when voltage is applied.

The ultrasound to be used is low-intensity pulsed ultrasound, and the ultrasound having a frequency of 20 kHz to 10 MHz, a burst width of 10 μsec to 1 msec, a repetition rate of 5 Hz to 10 kHz and an ultrasound intensity of 5-120 mW/cm² is expected to be effective for the differentiation induction. Ultrasound having a frequency of 1.5 MHz, a burst width of 200 μsec, a repetition rate of 1.0 kHz and an ultrasound intensity of 5-120 mW/cm² is preferable, and ultrasound having an ultrasound intensity of 30 mW is especially preferable.

The control means for controlling ultrasound is provided with an ultrasonic wave synthesizing function with which the ultrasound to be radiated from the ultrasound transducer is synthesized, an intensity controlling function, a storage arithmetic means and an on-off controlling function for electricity. These functions and means may be integrated with the main operating unit and built in it, or may be divided into the ultrasound transducer and the main operating unit.

In the present invention, a propylene tube is used as a vessel for undifferentiated cultured cell for convenience' sake of experiments, but the material and the shape of the vessel are not limited as far as they can be used for cell culture. In order to have the sure application of ultrasound, the ultrasound transducer and the culture vessel are stably placed in a water tank in such a state that they are in contact with each other.

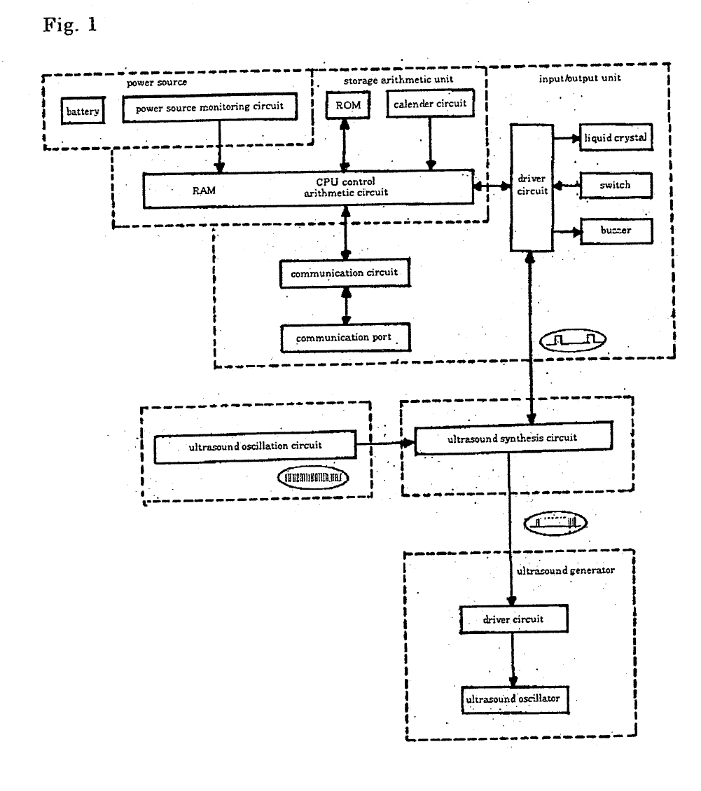

FIG. 1 is a block diagram which takes part in ultrasound irradiation in an apparatus for manufacturing artificial cartilage of the present invention. The block diagram is composed of a main operating unit comprising a power source, a storage arithmetic unit, an input/output unit, an oscillator, an ultrasonic wave synthesizing unit, and an ultrasound generator which has a built-in ultrasound transducer. The apparatus used in the examples is as follows: six units of the ultrasound generator are stably placed in the water tank with a frame; and ultrasound is applied to a polypropylene tube containing cultured human undifferentiated mesenchymal cells.

(1) Power Source:

A high capacity-type non-chargeable lithium battery is used. It has no power switch, the power is put on by the operation of the front panel, and the power is automatically turned off after the completion of an ultrasound irradiation for a specified time. The ultrasound generator is energized from the same battery.

The transmission of control signals of the ultrasound generator and the power supply are performed via an interconnection cable besides an operation switch, a liquid crystal display board and a buzzer.

(2) Storage Arithmetic Unit:

A CPU (central processing unit) is mounted on a printed board. The unit not only manages the irradiation time of ultrasound, but also monitors the operation and the state of the mounting of the ultrasound generator, and also performs a self-diagnosis. Further, the records of use are kept on a backup memory, and they can be taken out as required.

(3) Ultrasonic Wave Synthesizing Unit:

Signals having a burst width of pulsed ultrasound of 200 μsec and a repetition rate of 1 kHz are sent as the control signals for the ultrasound generator. Ultrasound signals of 1.5 MHz are produced in the oscillator. The control signals and the ultrasound signals of 1.5 MHz are combined to each other to produce electric signals of waveform having signal properties of a frequency of 1.5 MHz, an ultrasound intensity variable in the range of 0-150 mW/cm², a burst width of 200 μsec and a repetition rate of 1 kHz.

(4) Ultrasound Generator:

The ultrasound generator comprises a piezoelectric ceramic-made transducer, and it is connected to the main operation unit via a connection cable. A piezoelectric ceramic has such characteristics that voltage application generates microstrains. By using the properties, the electric signals produced by the ultrasonic wave synthesizing unit are given to the piezoelectric ceramic existing inside the transducer, and vibration (ultrasound) in its longitudinal direction is produced on the surface of the transducer. The vibration is used for irradiating chondrocytes. On the backside of the piezoelectric ceramic, an ultrasound absorber is adhered to prevent the leakage of the ultrasound.

### EXAMPLES

1. Examination of Cartilage Differentiation Inducing Activity Due to Ultrasound Irradiation

[Cell Culture]

Human undifferentiated mesenchymal cells were suspended in a 15 ml polypropylene tube, the suspension was subjected to centrifugation at 1,500 rpm for 5 min at room temperature, and they were allowed to stand at 37° C. for 24 hr under a 5%-CO₂ environment. Subsequently, the culture medium was exchanged, and the culture was continued. A cartilage-forming and differentiation-inducing basal medium (DMEM-high glucose, 0.17 mM of Ascorbic acid-2-phosphate, 0.35 mM of Proline, 1 mM of Sodium pyruvate, 0.1 μM of Dexamethasone, 6.25 μg/mL of Bovine insulin, 6.25 μg/mL of Transferrin, 6.25 μg/mL of Selenous acid, 5.33 μg/mL of Linoleic acid and 1.25 mg/mL of Bovine serum albumin) is used as the culture medium.

[Test Groups]

The cultured human undifferentiated mesenchymal cells were divided into the following three groups, and cartilage differentiation inducing activities were examined under ultrasound irradiation.

Group 1 (n=6): TGF-β3-free group.

Group 2 (n=6): cartilage differentiation inducing group with added TGF-β3.

Group 3 (n=6): cartilage differentiation inducing group with added TGF-β3 and ultrasound stimulation.

That is, Group 1 was an untreated group (TGF-β3-free group), Group 2 was a cartilage differentiation inducing group in which the cells were cultured in the presence of 10 ng/mL of TGF-β3, and Group 3 was a group in which the cells were cultured-under the same conditions as Group 2, but ultrasound was applied during culture. In each group, the test size was six (n=6), the culture was carried out at 37° C. under the atmosphere of 5% CO₂, and the cartilage-forming and differentiation-inducing basal medium was exchanged every 3 days.

[Conditions for Ultrasound Irradiation]

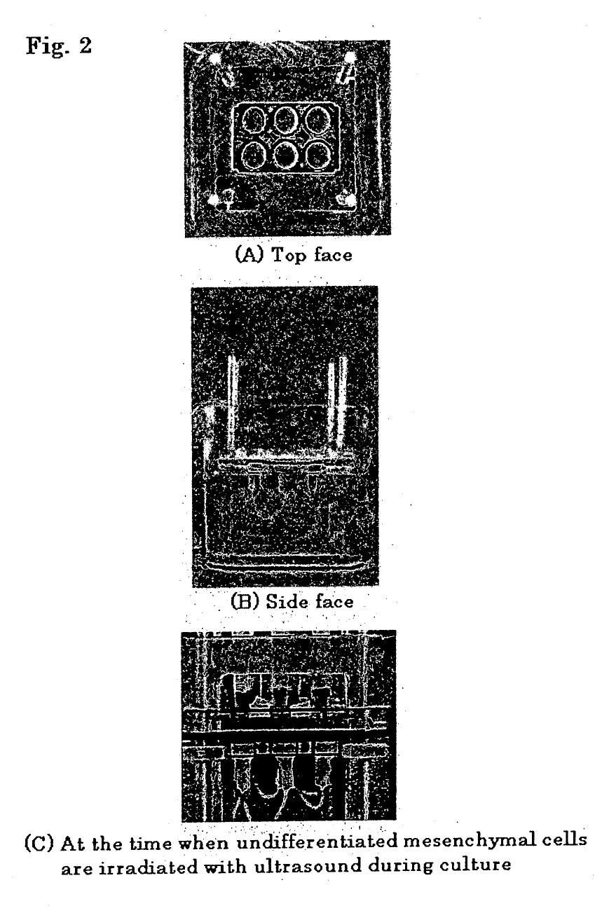

A polyethylene tube containing the cultured human undifferentiated mesenchymal cells of the ultrasound irradiation group (Group 3) was placed directly on the ultrasound generator (FIG. 2) installed in water, and pulsed ultrasound having a frequency of 1.5 kHz, a burst width of 200 μsec, a repetition rate of 1.0 kHz and an ultrasound intensity of 120 mW/cm² was applied to the polyethylene tube for 20 min a day every day for 10 days.

[Measuring Method]

(1) Measurement of Pellet Size

After 10 days of culture, pellets were photographed under a stereoscopic microscope (n=6). The pellet size was expressed by the maximum cross section.

(2) Histological Study

Histological study was performed by using pellets which had been treated by 48 hr fixation with 10% formalin, dehydration, paraffin embedding, alcian blue staining and anti-aggrecan antibody staining.

(3) Assays of DNA, Total Protein and Aggrecan

Pellets were washed with PBS twice and extracted with 4M guanidine hydrochloride at 4° C. for 24 hr, and the soluble extracted fraction was subjected to the assay of proteins and aggrecans. The extraction residue was subjected to the assaying of DNA after it had been treated with proteinase K. In the soluble extracted fraction, DNA was hardly detected.

[Result]

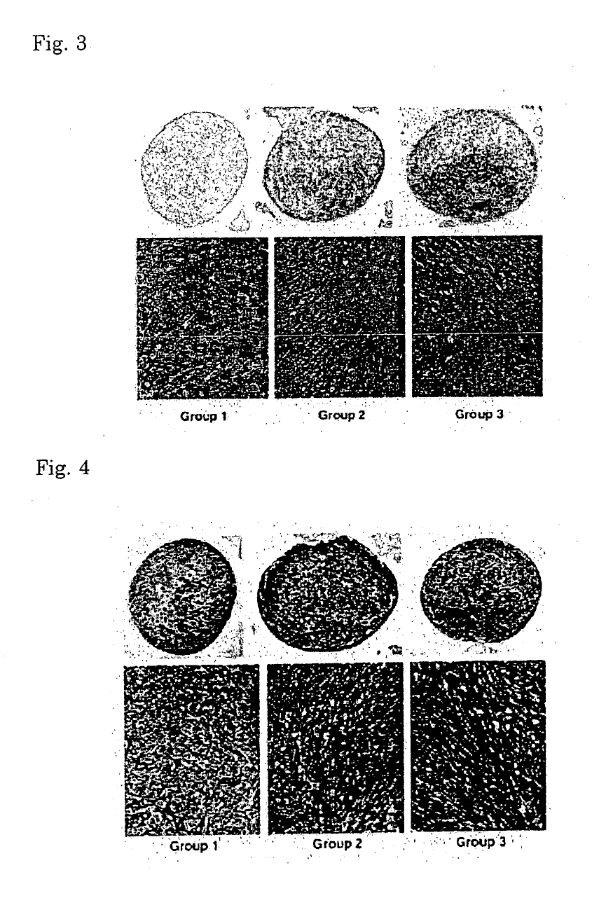

By the alcian blue staining (staining of acid mucopolysaccharide of cartilage substrate), pellets of Group 1 were slightly stained, but pellets of Group 2 and Group 3, which belong to differentiation inducing groups, were deeply stained. In the ultrasound stimulation group (Group 3), staining with high polarity was observed (FIG. 3).

In immunostaining with anti-aggrecan antibody, pellets of Group 1 were slightly stained, but pellets of Group 2 and Group 3 were stained deeply (FIG. 4). In the ultrasound stimulation group (Group 3), staining with high polarity was observed. The pattern of the immunostaining was same as that of the alcian blue staining.

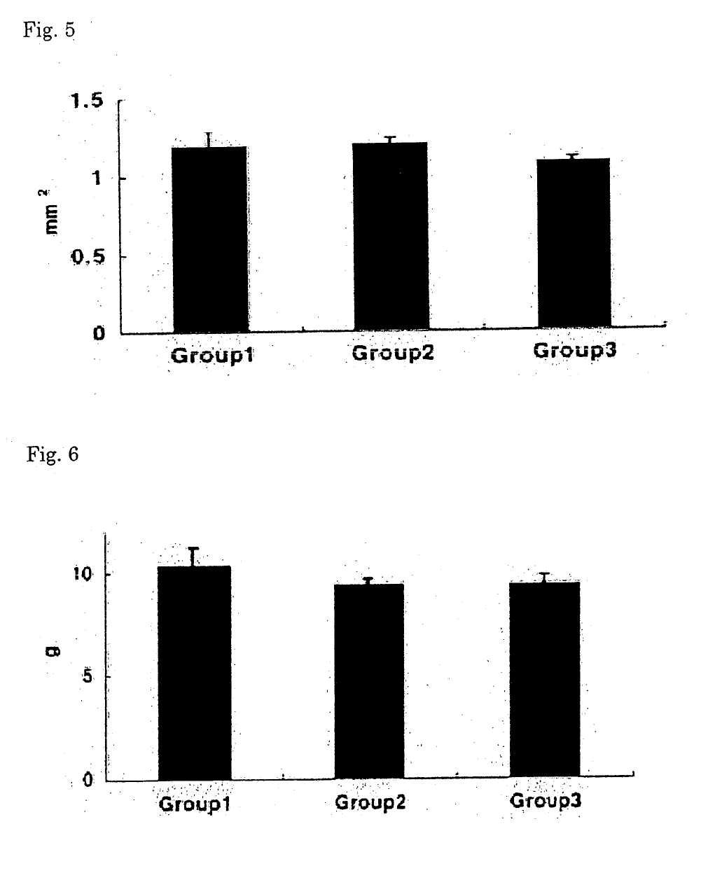

Pellet sizes were as follows: 1.19±0.10 mm² in Group 1; 1.19±0.05 mm² in Group 2; and 1.08±0.04 mm² in Group 3. The differentiation induction did not affect pellet size; but the pellet size was reduced by 7% in the ultrasound stimulation group (Group 3) (FIG. 5).

The amounts of DNA were as follows: 10.4±0.85 μg in Group 1; 9.36±0.28 μg in Group 2; and 9.38±0.42 μg in Group 3. In both cartilage differentiation inducing groups (Group 2 and Group 3), about 10% decrease in the amount of DNA was observed. No influence of ultrasound stimulation on DNA production was observed (FIG. 6).

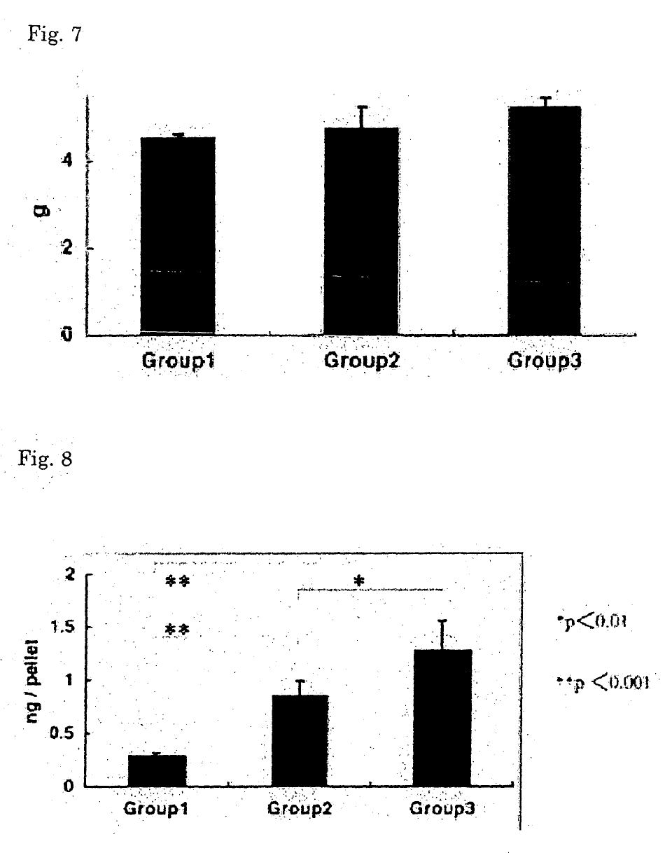

The amounts of total proteins were as follows: 4.52±0.06 μg in Group 1; 4.76±0.47 μg in Group 2; and 5.22±0.19 μg in Group 3. Compared with the TGF-β3-free group (Group 1), Group 2 and Group 3 had about 5% increase and about 15% increase, respectively, in the amount of total proteins (FIG. 7).

The amounts of aggrecans were as follows: 0.34±0.38 ng in Group 1; 1.38±0.76 ng in Group 2; and 2.55±1.15 ng in Group 3. The amount of aggrecans was increased about 4 times by the differentiation induction. Further, it was increased by 84% by the ultrasound stimulation (FIG. 8).

2. Examination of the Correlation of Ultrasound Intensities and Cartilage Differentiation Induction

By using the above-mentioned ultrasound irradiation apparatus, the correlation between ultrasound intensities and the amount of aggrecan as an index of differentiation induction activity was studied.

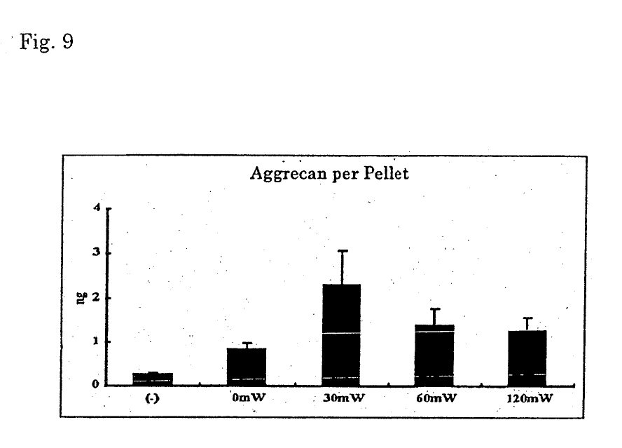

Ultrasound pulse having a frequency of 1.5 kHz, a burst width of 200 μsec and a repetition rate of 1.0 kHz, and an ultrasound intensity selected from 0, 30, 60 and 120 mW/cm² was applied for 20 min a day every day for 10 days to a polypropylene tube containing the cultured human undifferentiated mesenchymal cells of the ultrasound irradiation group (Group 3), wherein the polypropylene tube has been placed directly on the ultrasound generator installed in water. The result of the above mentioned control group (−) (Group 1) is also shown in FIG. 9.

The correlation between ultrasound intensities and the amount of aggrecan was studied, and it was found that a significant increase was observed in the ultrasound stimulation group (120 mW/cm², p<0.05) compared with the control group shown by (−) in the figure (0 mW/cm²). Further, the ultrasound intensity with which the largest increase in the amount of aggrecan was observed is 30 mW. (FIG. 9)

### EFFECT OF THE INVENTION

With ultrasound irradiation during the culture of undifferentiated mesenchymal cells collected from a patient himself in an in vitro culture system using a cartilage differentiation inducing medium, the present invention enables the preparation of an artificial cartilage in a shorter time than the conventional culture method.

In stead of the conventional method in which the chondrocytes having a limited supply source are implanted to a cartilage disorder site, it becomes possible that artificial cartilage is produced by using undifferentiated mesenchymal cells abundantly obtainable from the body of the patient himself and differentiating them quickly to chondrocytes, and the obtained chondrocytes can be used for cartilage restoration. By using this method, a rejection reaction, which always becomes a problem in allotransplantation, can be avoided.

1. A method for manufacturing artificial cartilage, characterized in that undifferentiated mesenchymal cells are cultured in a cartilage differentiation inducing medium, and the cells are irradiated with ultrasound.

2. A method for manufacturing artificial cartilage according to claim 1, characterized in that the ultrasound has a frequency of 20 kHz to 10 MHz, a burst width of 10 μsec to 1 msec, a repetition rate of 5 Hz to 10 kHz, and an ultrasound intensity of 5-120 mW/cm².

3. A method for manufacturing artificial cartilage according to claim 2, characterized in that the ultrasound has a frequency of 1.5 MHz, a burst width of 200 μsec, a repetition rate of 1.0 kHz, and an ultrasound intensity of 5-120 mW/cm².

4. An artificial cartilage that has been produced by ultrasound irradiation of undifferentiated mesenchymal cells under cultivation in a cartilage differentiation inducing medium.

5. An artificial cartilage that has been produced by irradiating undifferentiated mesenchymal cells under cultivation in a cartilage differentiation inducing medium with ultrasound having a frequency of 20 kHz to 10 MHz, a burst width of 10 μsec to 1 msec, a repetition rate of 5 Hz to 10 kHz, and an ultrasound intensity of 5-120 mW/cm².

6. A artificial cartilage according to claim 4, characterized in that the ultrasound has a frequency of 1.5 MHz, a burst width of 200 μsec, a repetition rate of 1.0 kHz, and an ultrasound intensity of 5-120 mW/cm².

7. An apparatus for manufacturing artificial cartilages comprising a culture vessel containing cultured undifferentiated mesenchymal cells, an ultrasound transducer for applying ultrasound to the vessel, a control means for controlling the ultrasound, and a holding water-tank for installing the ultrasound transducer and the culture vessel in the holding water-tank in such a state that they are in contact with each other.

8. An apparatus for manufacturing the artificial cartilage according to claim 7, characterized in that the control means is a means for controlling the ultrasound transducer so that ultrasound pulse is output from it.

9. An apparatus for manufacturing the artificial cartilage according to claim 8, characterized in that the ultrasound has a frequency of 20 kHz to 10 MHz, a burst width of 10 μsec to 1 msec, a repetition rate of 5 Hz to 10 kHz, and an ultrasound intensity of 5-120 mW/cm².

10. A method for accelerating cartilage differentiation induction, characterized in that undifferentiated mesenchymal cells are irradiated with ultrasound.

11. A method for accelerating cartilage differentiation induction according to claim 10, characterized in that the ultrasound has a frequency of 20 kHz to 10 MHz, a burst width of 10 μsec to 1 msec, a repetition rate of 5 Hz to 10 kHz, and an ultrasound intensity of 5-120 mW/cm².

12. A method for accelerating cartilage differentiation induction according to claim 10, characterized in that the ultrasound has a frequency of 1.5 MHz, a burst width of 200 μsec, a repetition rate of 1.0 kHz, and an ultrasound intensity of 5-120 mW/cm².
